(19) United States
(12) Patent Application Publication
(10) Pub. No.: US 2023/0036164 A1
(43) Pub. Date: Feb. 2, 2023

(54) BODY TEMPERATURE PREDICTION APPARATUS AND BODY TEMPERATURE PREDICTION METHOD, AND METHOD FOR TRAINING BODY TEMPERATURE PREDICTION APPARATUS

(71) Applicant: Research & Business Foundation SUNGKYUNKWAN UNIVERSITY, Suwon-si (KR)

(72) Inventors: Sukhan LEE, Suwon-si (KR); Chang Hoon SONG, Suwon-si (KR)

(73) Assignee: Research & Business Foundation SUNGKYUNKWAN UNIVERSITY, Suwon-si (KR)

(21) Appl. No.: 17/524,277

(22) Filed: Nov. 11, 2021

(30) Foreign Application Priority Data
Aug. 2, 2021 (KR) 10-2021-0101407

(51) Int. Cl. A61B 5/01; G06K 9/00; G06K 9/32; G06N 3/02

(52) U.S. Cl. CPC A61B 5/015; G06K 9/00248; G06K 9/00255; G06K 9/00281; G06K 9/3233; G06N 3/02

(57) ABSTRACT

An apparatus for predicting a body temperature is provided. The apparatus includes an external environment/activity estimation neural network configured to detect at least one facial region as a region of interest from an input thermal image of a target person to be measured, and estimate an environmental type including an external temperature and participation in physical activity based on a temperature of the at least one region of interest. The apparatus further includes a body temperature prediction neural network configured to predict a body temperature of the target person based on the environmental type estimated by the external environment/activity estimation neural network and the temperature of the at least one region of interest.

### CROSS-REFERENCE TO RELATED APPLICATION

This application claims priority to Korean Patent Application No. 10-2021-0101407, filed on Aug. 2, 2021. The entire contents of the application on which the priority is based are incorporated herein by reference.

### TECHNICAL FIELD

The present disclosure relates to a body temperature prediction apparatus and a body temperature prediction method, and a method for training the body temperature prediction apparatus, and more particularly, to an apparatus and a method capable of improving accuracy of body temperature prediction using a face temperature measuring apparatus.

### BACKGROUND

Recently, in order to prevent spread of infectious disease viruses and protect medical staff and managers, it is essential to check temperatures of visitors and patients. In particular, in order to prevent the spread of infectious diseases, a non-contact body temperature measuring apparatus that measures a skin temperature of a face with a thermal imaging camera is widely used.

In a case of using the non-contact body temperature measuring apparatus to measure a body temperature, a skin temperature is affected by an external environment and thus may be different than usual when a target person participates in external activities such as exercise. Thus, there is a problem that it is difficult to accurately predict the body temperature by measuring the skin temperature.

When the temperature of the skin, such as the face, wrist, and back of the hand among body parts for skin temperature measurement, is measured, the temperature of such a skin is not the same as the body temperature and may be 2 to 4° C. lower than the body temperature. Therefore, existing non-contact temperature measurement systems using thermal imaging cameras and infrared cameras are difficult to accurately predict the body temperature.

Therefore, there is high demand for a method that measures a skin temperature with a non-contact temperature measurement system while accurately predicting the body temperature in consideration of the skin temperature and external environment/ physical activities.

### SUMMARY

In view of the above, the present disclosure provides an apparatus and a method for accurately predicting a body temperature based on a measurement of a skin temperature taking into account an external environment and physical activity, and a method for accurately training the apparatus.

Technical objects to be achieved by the present disclosure are not limited to those described above, and other technical objects not mentioned above may also be clearly understood from the descriptions given below by those skilled in the art to which the present disclosure belongs.

In accordance with an aspect of the present disclosure, there is provided an apparatus for predicting a body temperature, the apparatus including: an external environment/activity estimation neural network configured to detect at least one facial region as a region of interest from an input thermal image of a target person to be measured, and estimate an environmental type including an external temperature and participation in physical activity based on a temperature of the at least one region of interest; and a body temperature prediction neural network configured to predict a body temperature of the target person based on the environmental type estimated by the external environment/activity estimation neural network and the temperature of the at least one region of interest.

Further, the external environment/activity estimation neural network may be trained by using, as input data for training, a temperature of the at least one region of interest for each of face thermal images of a plurality of training targets, and by using, as label data, an environmental type including an external temperature and participation in physical activity according to the temperature of the at least one region of interest when measuring the temperature for each of the plurality of training targets.

Further, the body temperature prediction neural network may be trained by using, as input data for training, a temperature of the at least one region of interest for each of face thermal image images of the plurality of training targets and an environmental type for training including an external temperature and participation in physical activity according to the temperature of the at least one region of interest for each of the plurality of training targets, and by using, as a label data, a body temperature obtained when measuring the temperature for each of the training targets.

Further, the at least one facial region may include inner sides of eyes, a nose, and a cheek.

Further, the environmental type may include at least one of a hot environment, an environment with exercise, a normal environment without exercise, and a cold environment.

Further, in the detected region of interest, a face region may be detected from the input thermal image of the target person based on a first object detection algorithm, and the at least one facial region may be detected as the region of interest within the detected face region based on a second object detection algorithm.

In accordance with another aspect of the present disclosure, there is provided a method for predicting a body temperature, the method including: detecting at least one facial region as a region of interest from an input thermal image of a target person to be measured, and estimating an environmental type including an external temperature and participation in physical activity based on a temperature of the at least one region of interest; and predicting a body temperature of the target person based on the estimated environmental type and the temperature of the at least one region of interest for the input thermal image.

Further, the at least one facial region may include inner sides of eyes, a nose, and a cheek.

Further, the environmental type may include at least one of a hot environment, an environment with exercise, a normal environment without exercise, and a cold environment.

Further, in the detected region of interest, a face region is detected from the input thermal image of the target person based on a first object detection algorithm, and the at least one facial region is detected as the region of interest within the detected face region based on a second object detection algorithm.

In accordance with still another aspect of the present disclosure, there is provided a method for training a body temperature prediction apparatus, the method including: training an external environment/activity estimation neural network by using, as first input data for training, a plurality of face thermal images for training and by using, as first label data, an environmental type including an external temperature and participation in physical activity according to a temperature of at least one facial region, which is a region of interest, of each of the plurality of face thermal images for training to detect the at least one region of interest of a target person from an input thermal image of the target person, and estimate the environmental type for the target person based on the temperature of the at least one region of interest for the target person; and training a body temperature prediction neural network by using, as second input data for training, a plurality of face thermal images for training and a plurality of estimated environmental types for training including an external temperature and participation in physical activity and by using, as second label data, body temperatures obtained based on a temperature of at least one facial region, which is a region of interest, of each of the plurality of face thermal images for training and the plurality of estimated environmental types for training to predict a body temperature of the target person based on the temperature of the at least one region of interest of the target person and the estimated environmental type for the target person.

According to the embodiments of the present disclosure, a body temperature is accurately predicted through a deep learning network based on the skin temperature measurement and the external environment/activities. Therefore, it becomes possible to thoroughly manage people entering and leaving places with a large floating population such as hospitals and airports that are sensitive to fever symptoms. In addition, it becomes possible to accurately predict the body temperature through non-contact face temperature measurement, thereby preventing the spread of infectious diseases and quickly predicting the body temperature.

### BRIEF DESCRIPTION OF THE DRAWINGS

FIG. 1 is a block diagram illustrating a body temperature prediction apparatus according to one embodiment of the present disclosure.

FIGS. 2A and 2B illustrate face thermal images showing a facial region having a highest temperature among facial regions and a method for measuring a temperature of a face region of interest (ROI) according to one embodiment of the present disclosure.

FIGS. 3A to 3C are graphs each illustrating a correlation between a facial region and body temperature data according to various environments and physical activities.

FIGS. 4A to 4C are graphs each illustrating a correlation between facial regions according to various environments and physical activities.

FIG. 5 is a graph illustrating a body temperature prediction result for a model that predicts a body temperature by using a temperature of a region of interest of the facial region as input data.

FIG. 6 is a graph illustrating a body temperature prediction result for a model that predicts a body temperature by using a temperature of a region of interest of the facial region and an environmental type including an external temperature and participation in physical activity as input data according to one embodiment of the present disclosure.

FIG. 7 is a block diagram for describing a body temperature prediction apparatus according to one embodiment of the present disclosure in terms of hardware.

FIG. 8 is a flowchart of a method for training a body temperature prediction apparatus according to one embodiment of the present disclosure.

### DETAILED DESCRIPTION

The advantages and features of exemplary embodiments of the present disclosure and methods of accomplishing them will be clearly understood from the following description of the embodiments taken in conjunction with the accompanying drawings. However, the present disclosure is not limited to those embodiments and is implemented in various forms. It is noted that the embodiments are provided to make a full disclosure and also to allow those skilled in the art to know the full scope of the present disclosure.

In the following description, well-known functions and/or configurations will not be described in detail if they would unnecessarily obscure the features of the disclosure. Further, the terms to be described below are defined in consideration of their functions in the embodiments of the disclosure and vary depending on a user’s or operator’s intention or practice. Accordingly, the definition is made on a basis of the content throughout the present disclosure.

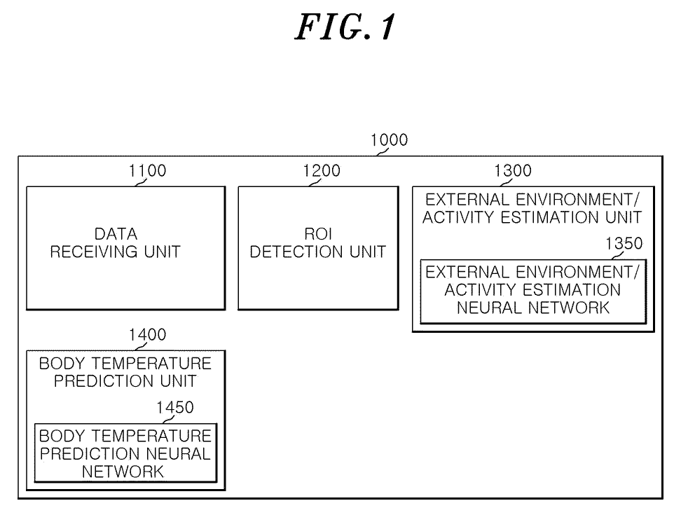

FIG. 1 is a block diagram illustrating a body temperature prediction apparatus according to one embodiment of the present disclosure.

Referring to FIG. 1, a body temperature prediction apparatus 1000 may include a data receiving unit 1100, a region of interest (ROI) detection unit 1200, an external environment/activity estimation unit 1300, and a body temperature prediction unit 1400.

In the present disclosure, for the sake of convenience of descriptions, it is described that the external environment/activity estimation unit 1300 and the body temperature prediction unit 1400 are included in the body temperature prediction apparatus 1000. However, the present disclosure is not limited thereto. For example, depending on an embodiment, the external environment/activity estimation unit 1300 and the body temperature prediction unit 1400 may be separately provided or may be executed as programs stored in a storage unit.

The body temperature prediction apparatus 1000 may estimate an environmental type including an external environment and participation in physical activity (for example, whether the physical activity has been performed or not) from an input thermal image by using a pre-trained external environment/activity estimation neural network 1350.

Moreover, the body temperature prediction apparatus 1000 may use a pre-trained body temperature prediction neural network 1450 to finally predict the body temperature by using the input thermal image and the estimated environmental type as inputs.

The data receiving unit 1100 may internally or externally receive a thermal image of the body. For example, the data receiving unit 1100 may receive a face thermal image or a full-body thermal image of a body from an external or internal imaging device.

The ROI detection unit 1200 may detect the face ROI in the thermal image received from the data receiving unit 1100 and receive a temperature of the selected face ROI.

In more detail, the ROI detection unit 1200 may detect a preset face ROI region in the received thermal image by using an object detection method. The object detection method may include regions with convolutional neural network (R-CNN), a single shot multi-box detector (SSD), you only look once (YOLO), or the like.

According to one embodiment of the present disclosure, the ROI detection unit 1200 may first detect a face in the received thermal image using the YOLO object detection method. Subsequently, the ROI detection unit 1200 may detect a preset face ROI from the detected face using a second YOLO object detection method. Accordingly, the ROI detection unit 1200 may acquire a temperature for the detected face ROI.

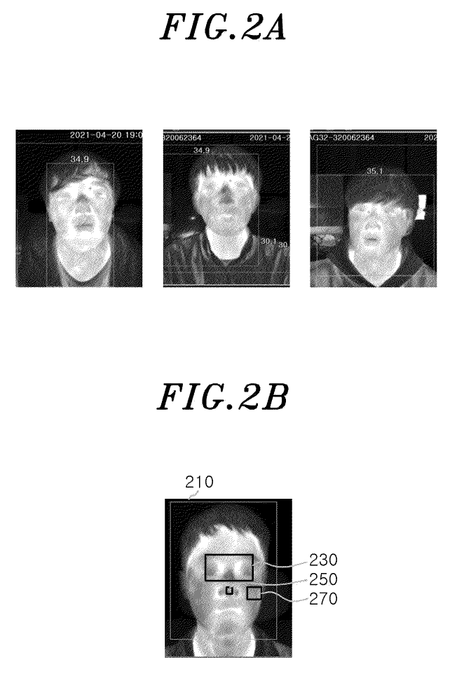

FIG. 2A is face thermal images showing a facial region having the highest temperature among human facial regions, and FIG. 2B illustrates a method for measuring the temperature of the facial ROI according to one embodiment of the present disclosure.

As shown in FIG. 2A, a facial region having the highest temperature among the human facial regions is inner sides of eyes around eye canthi. A human body temperature is generally higher than a skin temperature. Accordingly, a change in body temperature may be detected by selecting, as the face ROI, the facial region that has the highest temperature among the temperatures of the facial skin.

According to one embodiment of the present disclosure, the ROI detection unit 1200 may select, as the face ROI, the inner sides of the eyes, which are the facial regions at which the highest temperature is measured among the temperatures of the facial skin. In addition, in order to estimate the external environment, a nose and a cheek can be additionally selected as the face ROIs.

Referring to FIG. 2B, according to one embodiment of the present disclosure, by using the YOLO object detection method, the ROI detection unit 1200 detects the human face region 210 and further detects the regions of the inner sides of the eyes 230, the nose 250, and the cheek 270 that are preset as the face ROIs from the detected face region.

Then, the ROI detection unit 1200 acquires the temperatures of all pixels in each of the detected face ROI regions. The ROI detection unit 1200 may determine the highest temperature among the acquired temperatures of all pixels in the region of the inner sides of the eyes 230 as the representative temperature of the inner sides of the eyes 230. Moreover, the ROI detection unit 1200 may determine an average value of the temperatures of all pixels in each of the region of the nose 250 and the region of the cheek 270 as the representative temperature of each of the nose 250 and the cheeks 270. Since the temperature of the inner sides of the eyes 230 is the most similar to the body temperature, the highest temperature among the pixel temperatures in the region of the inner sides of the eyes 230 may be used as the representative temperature. The nose 250 and the cheek 270 are facial regions having the temperatures that are sensitively changed according to the external environment and physical activity, and the temperature fluctuation range is large depending on the pixels in the region. Thus, the average temperature of all pixels in each of the regions of the nose 250 and the cheek 270 may be used as the representative temperature for estimating the environmental type including the external environment and participation in the physical activity.

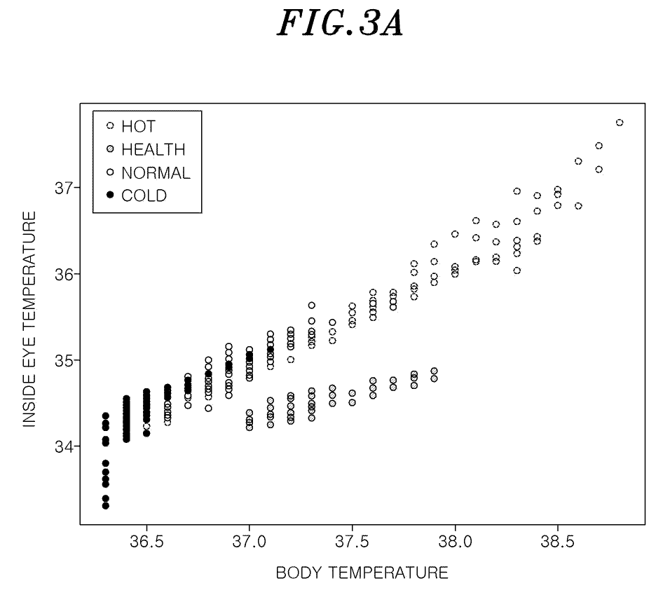

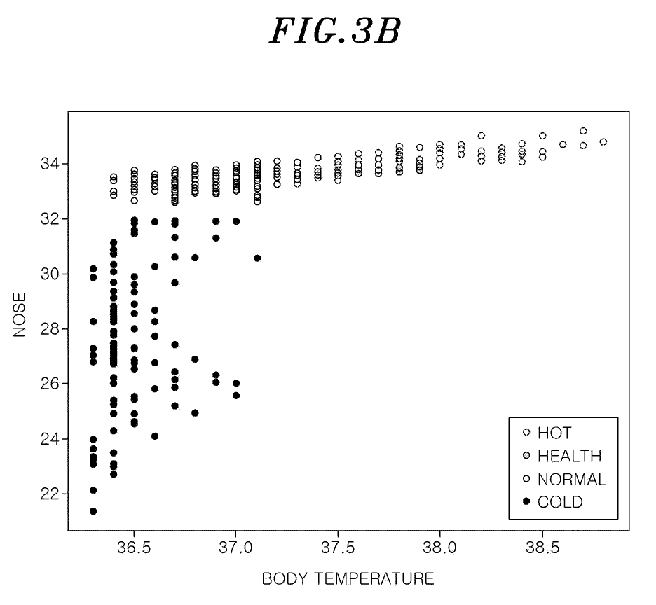

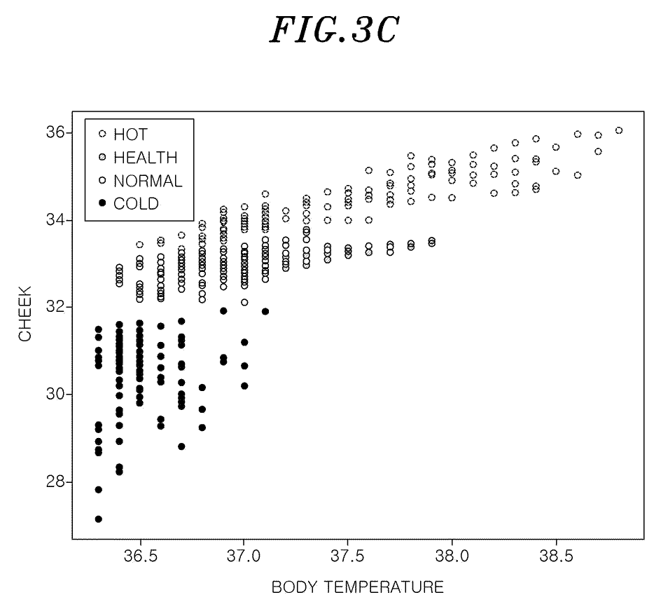

FIGS. 3A to 3C are graphs each illustrating a correlation between a facial region and body temperature data according to various environments and activities.

Referring to FIGS. 3A to 3C, there exists a correlation between the body temperature and the temperatures of face ROIs such as the inner sides of eyes around eye canthi, the nose and the cheek that varys according to the external environment and the physical activities. This correlation represents a bases for embodiment of the present disclosure.

Specifically, as shown in FIG. 3A in which an x-axis indicates the body temperature and a y-axis indicates the temperature of the inner sides of the eyes (inside eye temperature),a high correlation is observed between the body temperature and the temperature of the inner sides of the eyes since the temperature of the inner sides of the eyes increase as the body temperature increases in a hot environment (hot), an environment with exercise (health), and a normal environment without exercise (normal). However, in a cold environment (cold), the body temperature does not drop below 36° C. whereas it can be seen that the temperature of the inner sides of the eyes is lowered due to the skin being affected by the cold environment.

Referring to FIGS. 3B and 3C, similar to the relationship between the temperature of the inner sides of the eyes and the body temperature, the relationships between the body temperature on the x-axis and the temperature of the nose on the y-axis and between the body temperature on the x-axis and the temperature of the cheek on the y-axis show the body temperature does not drop below 36° C. in the cold environment (cold) whereas each of the temperature of the nose and the temperature of the cheek is lowered due to the skin being affected by the cold environment. However, when the temperatures of the nose and the cheek are measured after the nose and the cheek are being in the cold environment, it can be confirmed that even when the body temperature increases, the temperatures of the nose and the cheek do not increase rapidly compared to the temperature of the inner sides of the eyes because the skin of the nose and the cheek is thicker than the skin around the inner sides of the eyes.

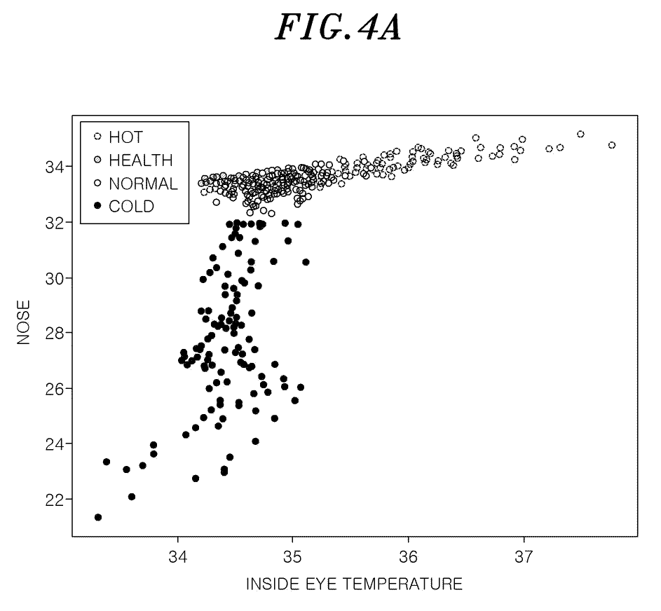

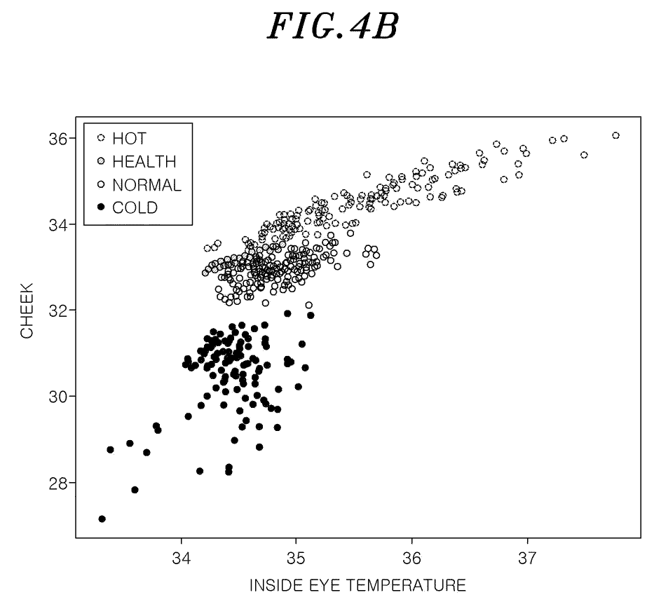

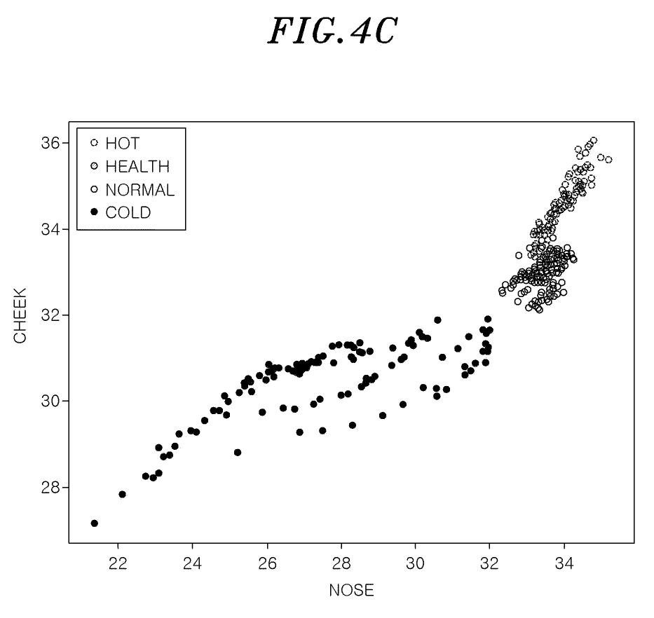

FIGS. 4A to 4C are graphs each illustrating a correlation between facial regions according to various environments and physical activities.

Referring to FIGS. 4A to 4C, the correlations according to various environments and physical activities are observed among the temperature of the inner sides of the eyes, the temperature of the nose, and the temperature of the cheek that are collected to predict the body temperature. As the temperature of the inner sides of the eyes around canthi of eyes increases, the temperatures of the nose and the cheek also tend to increase. However, the temperature of the nose and the cheek tends to remain relatively same except the case where they are exposed to a cold environment.

Referring back to FIG. 1, the external environment/activity estimation unit 1300 may include the external environment/activity estimation neural network 1350, and obtains the temperatures of the face ROIs from the ROI detection unit 1200 to estimate the environmental type including the external environment and the participation in physical activity.

According to one embodiment, the temperatures of the face ROIs may include the temperature of the inner sides of the eyes, the temperature of the nose, and the temperature of the cheek, and the environmental type may include the cold environment, the hot environment, the environment with exercise, and the normal environment without exercise.

Specifically, the external environment/activity estimation neural network 1350 may be an artificial neural network model which is trained by using, as input data for training, a temperature of at least one facial region in a face thermal image of each of the plurality of training targets (e.g., the plurality of target persons to be measured), and by using, as label data, the environmental type according to the temperature of the at least one facial region when measuring the temperature of the at least one facial region for each of the plurality of training targets.

The external environment/activity estimation unit 1300 receives the temperature of the inner sides of the eyes, the temperature of the nose, and the temperature of the cheek of a target person to be measured, and uses the external environment/activity estimation neural network 1350 to obtain estimated respective probabilities for the cold environment, the hot environment, the environment with exercise, and the normal environment without exercise using a softmax function.

The body temperature prediction unit 1400 may include the body temperature prediction neural network 1450, and obtains the temperatures of the face ROIs from the ROI detection unit 1200 and the probability of each environmental type estimated from the external environment/activity estimation unit 1300 to predict the body temperature of the target person.

Specifically, according to one embodiment, the body temperature prediction neural network 1450 may be an artificial neural network model which is trained by using, as input data for training, the temperature of the at least one facial region in the face thermal image of each of the plurality of training targets and the environmental type for training when measuring the temperature of the at least one facial region for each of the plurality of training targets, and by using, as label data, the body temperature obtained when measuring the temperature of the at least one facial region for each of the plurality of training targets.

That is, the body temperature prediction unit 1400 receives the temperature of the inner sides of the eyes, the temperature of the nose, and the temperature of the cheek of the target person and the estimated probabilities for the cold environment, the hot environment, the environment with exercise, and the normal environment without exercise that are obtained from the external environment/activity neural network 1350. Then, the body temperature prediction unit 1400 predicts the body temperature of the target person by using the trained body temperature prediction neural network 1450.

In this specification, for the sake of convenience of explanation, the external environment/activity estimation unit 1300 and the body temperature prediction unit 1400 are described in a functionally separated form, but may perform the functions as an integrated artificial neural network.

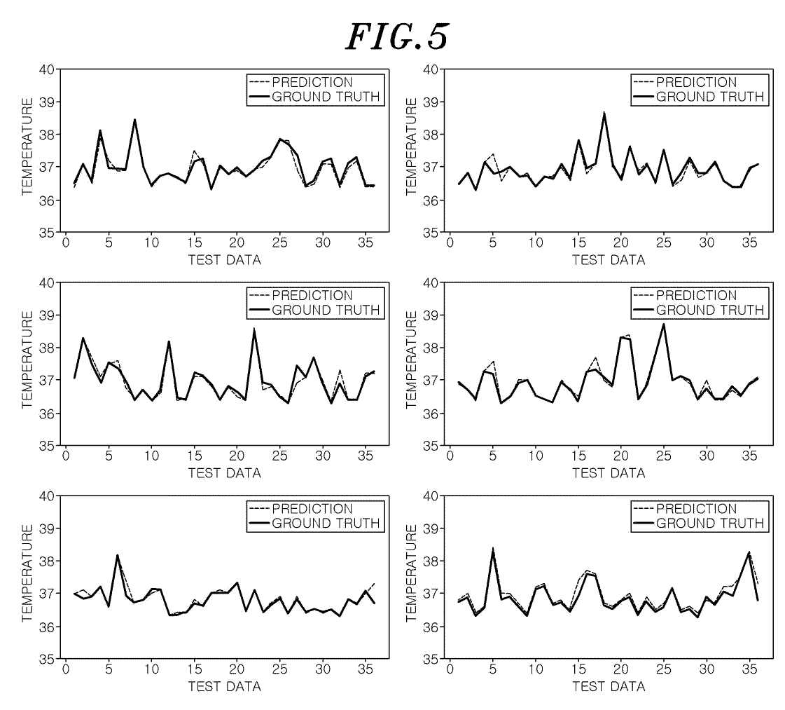

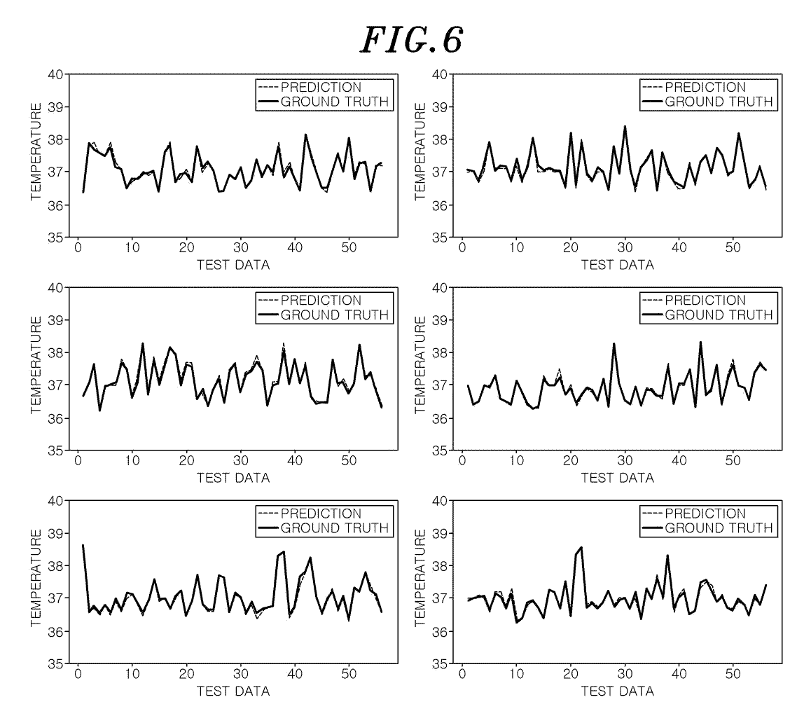

FIG. 5 is a graph illustrating a body temperature prediction result for a model that predicts body temperature using the temperature of the region of interest of the facial region as input data.

Referring to FIG. 5, 36 data sets for an actual body temperature and a predicted body temperature were used for each experiment in the body temperature prediction model, and six experiments were performed to compare the experiments with each other. The body temperature prediction model used in the experiment is an artificial neural network model trained by using, as input data for training, a plurality of temperatures of the face ROIs for training including temperatures of inner sides of the eyes, temperatures of the nose, and temperatures of the cheek, and by using a plurality of actual body temperature data as label data.

As shown in the graphs of FIG. 5, when the predicted body temperature (Prediction) and the actual body temperature (Ground Truth) are compared with each other after a temperature of the inner sides of the eyes, a temperature of the nose, and a temperature of the cheek are input to the body temperature prediction model trained for the body temperature prediction, the predicted body temperature and the actual body temperature are similar to each other. A mean square error between the predicted body temperature and the actual body temperature is 0.0499, and a large difference between the predicted body temperature and the actual body temperature appears in the environment with exercise (environment after the completion of the exercise).

FIG. 6 is a graph illustrating a body temperature prediction result for a model that predicts a body temperature using a temperature of at least one region of interest of the facial region and an environmental type including an external temperature and participation in physical activity as input data according to one embodiment of the present disclosure.

Referring to FIGS. 1 and 6, according to one embodiment of the present disclosure, 36 data sets for the actual body temperature and the predicted body temperature were used for each experiment in the body temperature prediction model, and six experiments were performed to compare the experiments with each other. The body temperature prediction model used for body temperature prediction according to the embodiment is an artificial neural network model in which the external environment/activity estimation neural network 1350 of the external environment/activity estimation unit 1300 and the body temperature prediction neural network 1450 of the body temperature prediction unit 1400 are connected to each other to predict the body temperature.

As shown in the graphs of FIG. 6, when the predicted body temperature (Prediction) and the actual body temperature (Ground Truth) are compared with each other after a temperature of the inner sides of the eyes, a temperature of the nose, and a temperature of the cheek are input to the body temperature prediction model trained for the body temperature prediction, the predicted body temperature and the actual body temperature are much more similar to each other compared to the body temperature prediction model used in FIG. 5. Further, the mean square error between the predicted body temperature and the actual body temperature is 0.0033, and it can be confirmed that the body temperature prediction model according to the embodiment of the present disclosure can measure the body temperature more accurately in all environmental types, compared to the body temperature prediction model used in FIG. 5.

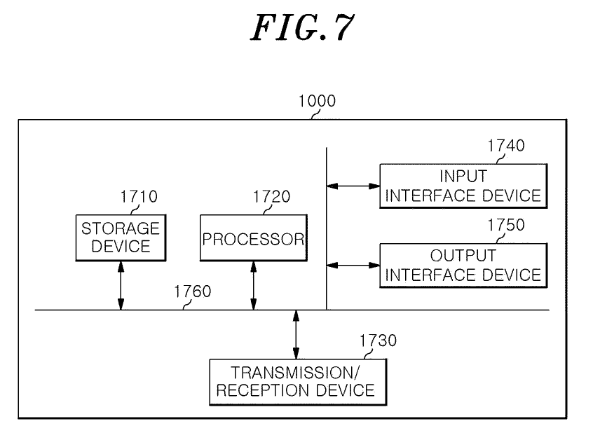

FIG. 7 is a block diagram for describing the body temperature prediction apparatus according to one embodiment of the present disclosure in terms of hardware.

Referring to FIGS. 1 and 7, the body temperature prediction apparatus 1000 may include a storage device 1710 that stores one or more commands (computer-executable instructions), a processor 1720 that executes the one or more commands in the storage device 1710, a transmission/reception device 1730, an input interface device 1740, and an output interface device 1750.

The above described components 1710, 1720, 1730, 1740, and 1750 included in the body temperature prediction apparatus 1000 may be connected by a data bus 1760 to communicate with each other.

The storage device 1710 may include a memory or at least one of a volatile storage medium and a non-volatile storage medium. For example, the storage device 1710 may include at least one of a read only memory (ROM) and a random access memory (RAM) .

The storage device 1710 may further include one or more command (instructions) to be executed by the processor 1720 to be described below.

According to one embodiment of the present disclosure, one or more command (instructions) to be executed by the processor 1720 may include a first command and a second command. Specifically, the first command is used to train the external environment/activity estimation neural network 1305 by using a plurality of face thermal images for training as first training input data and by using, as first label data, an environmental type including an external environment and participation in physical activity according to a temperature of at least one facial region, which is a region of interest, of each of the plurality of face thermal images for training to detect the at least one region of interest of a target person from an input thermal image of the target person, and estimate the environmental type for the target person based on the temperature of the at least one region of interest for the target person. The second command is used to train the body temperature prediction neural network 1450 by using a plurality of face thermal images for training and a plurality of estimated environmental types for training as second training input data and by using, as second label data, body temperatures obtained based on a temperature of at least one facial region, which is a region of interest, of each of the plurality of face thermal images for training and the plurality of estimated environmental types for training to predict a body temperature of the target person based on the temperature of the at least one region of interest of the target person and the estimated environmental type for the target person.

The processor 1720 may include a central processing unit (CPU), a graphics processing unit (GPU), a micro controller unit (MCU), or a dedicated processor that executes methods according to the embodiments of the present disclosure.

Referring further to FIG. 1, as described above, the processor 1720 may execute the functions of the ROI detection unit 1200, the external environment/activity estimation unit 1300, and the body temperature prediction unit 1400 by one or more commands stored in the storage device 1710, and each of the ROI detection unit 1200, the external environment/activity estimation unit 1300, and the body temperature prediction unit 1400 may be stored in a memory in the form of at least one module to be executed by the processor.

The input interface device 1740 may receive at least one control signal from a user. In addition, the input interface device 1740 may perform the function of the data receiving unit 1100 that receives the thermal image of the target person captured by an external device.

The output interface device 1750 may output and visualize at least one piece of information including the predicted body temperature of the target person by the operation of the processor 1720.

In the above description, the body temperature prediction apparatus according to one embodiment of the present disclosure has been described. Hereinafter, a body temperature prediction method according to one embodiment of the present disclosure will be described. The body temperature prediction method is executed by an operation of the processor in the body temperature prediction apparatus.

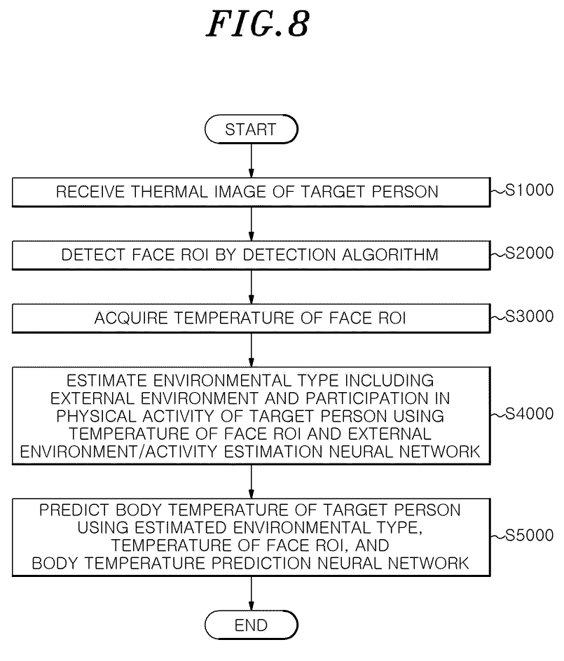

FIG. 8 is a flowchart of a method for training a body temperature prediction apparatus according to one embodiment of the present disclosure.

Referring to FIGS. 1, 7, and 8, the transmission/reception device 1730 in the body temperature prediction apparatus 1000 may receive the thermal image of a target person to be measured from the outside (step S1000).

Thereafter, the processor 1720 first detects a face of the target person from the received thermal image of the target person by using an object detection algorithm including the YOLO object detection method, and then uses the object detection algorithm again to detect face ROIs (step S2000). Then, the processor 1720 acquires a temperature of each of the face ROIs (step S3000).

Subsequently, the processor 1720 may estimate an environmental type including an external environment and participation in physical activity of the target person by using the pre-trained external environment/activity estimation neural network 1350 with the acquired temperature of each of the face ROIs as input data (step S4000).

Finally, the processor 1720 may predict the body temperature of the target person by using the pre-trained body temperature prediction neural network 1450 with the acquired temperature of each of the face ROIs and the estimated environmental type of the target person as input data (step S5000) .

According to the above embodiment of the present disclosure, there may be provided a method for predicting a body temperature, the method including detecting at least one facial region as a region of interest from an input thermal image of a target person to be measured, estimating an environmental type including an external temperature and participation in physical activity based on a temperature of the at least one region of interest, and predicting a body temperature of the target person based on the estimated environmental type and the temperature of the at least one region of interest for the input thermal image.

Therefore, according to the embodiments of the present disclosure, it is possible to provide the apparatus and the method that can estimate the environmental type of the target person and predict the body temperature using the estimated result (the estimated environmental type of the target person) and the temperatures of the face ROIs. Accordingly, even when the facial skin is affected by various external environmental and physical activities, it is possible to accurately predict the body temperature.

Further, since the apparatus and method according to the embodiments of the present disclosure can accurately predict the body temperature of the target person to be measured in a non-contact manner, the apparatus and method according to the embodiment of the present disclosure can be effective in preventing infectious diseases and preventing the spread of infectious diseases.

The combinations of respective blocks of block diagrams and respective sequences of a flow diagram attached herein is carried out by computer program instructions which are executed through various computer means and stored in a non-transitory computer-readable storage medium. Since the computer program instructions is loaded in processors of a general purpose computer, a special purpose computer, or other programmable data processing apparatus, the instructions, carried out by the processor of the computer or other programmable data processing apparatus, create means for performing functions described in the respective blocks of the block diagrams or in the respective sequences of the sequence diagram. Since the computer program instructions, in order to implement functions in specific manner, is stored in a memory unit, which comprises non-transitory computer-readable medium, useable or readable by a computer or a computer aiming for other programmable data processing apparatus, the instruction stored in the memory unit useable or readable by a computer produces manufacturing items including an instruction means for performing functions described in the respective blocks of the block diagrams and in the respective sequences of the sequence diagram. Since the computer program instructions are loaded in a computer or other programmable data processing apparatus, instructions, a series of sequences of which is executed in a computer or other programmable data processing apparatus to create processes executed by a computer to operate a computer or other programmable data processing apparatus, provides operations for executing functions described in the respective blocks of the block diagrams and the respective sequences of the flow diagram. The computer program instructions are also performed by one or more processes or specifically configured hardware (e.g., by one or more application specific integrated circuits or ASIC(s)). The non-transitory computer-readable recording medium includes, for example, a program command, a data file, a data structure and the like solely or in a combined manner. The program command recorded in the medium is a program command specially designed and configured for the present disclosure or a program command known to be used by those skilled in the art of the computer software. The non-transitory computer-readable recording medium includes, for example, magnetic media, such as a hard disk, a floppy disk and a magnetic tape, optical media, such as a CD-ROM and a DVD, magneto-optical media, such as a floptical disk, and hardware devices specially configured to store and execute program commands, such as a ROM, a RAM, a flash memory and the like. The program command includes, for example, high-level language codes that can be executed by a computer using an interpreter or the like, as well as a machine code generated by a compiler. The hardware devices can be configured to operate using one or more software modules in order to perform the operation of the present disclosure, and vice versa. In some embodiments, one or more of the processes or functionality described herein is/are performed by specifically configured hardware (e.g., by one or more application specific integrated circuits or ASIC(s)). Some embodiments incorporate more than one of the described processes in a single ASIC. In some embodiments, one or more of the processes or functionality described herein is/are performed by at least one processor which is programmed for performing such processes or functionality.

Moreover, the respective blocks or the respective sequences in the appended drawings indicate some of modules, segments, or codes including at least one executable instruction for executing a specific logical function(s). In several alternative embodiments, it is noted that the functions described in the blocks or the sequences run out of order. For example, two consecutive blocks and sequences are substantially executed simultaneously or often in reverse order according to corresponding functions.

The explanation as set forth above is merely described a technical idea of the exemplary embodiments of the present disclosure, and it will be understood by those skilled in the art to which this disclosure belongs that various changes and modifications is made without departing from the scope and spirit of the claimed invention as disclosed in the accompanying claims. Therefore, the exemplary embodiments disclosed herein are not used to limit the technical idea of the present disclosure, but to explain the present disclosure. The scope of the claimed invention is to be determined by not only the following claims but also their equivalents. Specific terms used in this disclosure and drawings are used for illustrative purposes and not to be considered as limitations of the present disclosure. Therefore, the scope of the claimed invention is construed as defined in the following claims and changes, modifications and equivalents that fall within the technical idea of the present disclosure are intended to be embraced by the scope of the claimed invention.

What is claimed is:

1. An apparatus for predicting a body temperature, the apparatus comprising:
an external environment/activity estimation neural network configured to detect at least one facial region as a region of interest from an input thermal image of a target person to be measured, and estimate an environmental type including an external temperature and participation in physical activity based on a temperature of the at least one region of interest; and
a body temperature prediction neural network configured to predict a body temperature of the target person based on the environmental type estimated by the external environment/activity estimation neural network and the temperature of the at least one region of interest.

2. The apparatus of claim 1, wherein the external environment/activity estimation neural network is trained by using, as input data for training, a temperature of the at least one region of interest for each of face thermal images of a plurality of training targets, and by using, as label data, an environmental type including an external temperature and participation in physical activity according to the temperature of the at least one region of interest when measuring the temperature for each of the plurality of training targets.

3. The apparatus of claim 1, wherein the body temperature prediction neural network is trained by using, as input data for training, a temperature of the at least one region of interest for each of face thermal image images of the plurality of training targets and an environmental type for training including an external temperature and participation in physical activity according to the temperature of the at least one region of interest for each of the plurality of training targets, and by using, as a label data, a body temperature obtained when measuring the temperature for each of the training targets.

4. The apparatus of claim 1, wherein the at least one facial region includes inner sides of eyes, a nose, and a cheek.

5. The apparatus of claim 1, wherein the environmental type includes at least one of a hot environment, an environment with exercise, a normal environment without exercise, and a cold environment.

6. The apparatus of claim 1, wherein in the detected region of interest, a face region is detected from the input thermal image of the target person based on a first object detection algorithm, and the at least one facial region is detected as the region of interest within the detected face region based on a second object detection algorithm.

7. A method for predicting a body temperature, the method comprising:
detecting at least one facial region as a region of interest from an input thermal image of a target person to be measured, and estimating an environmental type including an external temperature and participation in physical activity based on a temperature of the at least one region of interest; and
predicting a body temperature of the target person based on the estimated environmental type and the temperature of the at least one region of interest for the input thermal image.

8. The method of claim 7, wherein the at least one facial region includes inner sides of eyes, a nose, and a cheek.

9. The method of claim 7, wherein the environmental type includes at least one of a hot environment, an environment with exercise, a normal environment without exercise, and a cold environment.

10. The method of claim 7, wherein in the detected region of interest, a face region is detected from the input thermal image of the target person based on a first object detection algorithm, and the at least one facial region is detected as the region of interest within the detected face region based on a second object detection algorithm.

11. A method for training a body temperature prediction apparatus, the method comprising:
training an external environment/activity estimation neural network by using, as first training input data, a plurality of face thermal images for training and by using, as first label data, an environmental type including an external temperature and participation in physical activity according to a temperature of at least one facial region, which is a region of interest, of each of the plurality of face thermal images for training to detect the at least one region of interest of a target person from an input thermal image of the target person, and estimate the environmental type for the target person based on the temperature of the at least one region of interest for the target person; and
training a body temperature prediction neural network by using, as second training input data, a plurality of face thermal images for training and a plurality of estimated environmental types for training including an external temperature and participation in physical activity and by using, as second label data, body temperatures obtained based on a temperature of at least one facial region, which is a region of interest, of each of the plurality of face thermal images for training and the plurality of estimated environmental types for training to predict a body temperature of the target person based on the temperature of the at least one region of interest of the target person and the estimated environmental type for the target person.

12. The method of claim 11, wherein the at least one facial region includes inner sides of eyes, a nose, and a cheek.

13. The method of claim 11, wherein the environmental type includes at least one of a hot environment, an environment with exercise, a normal environment without exercise, and a cold environment.

14. The method of claim 11, wherein in the detected region of interest, a face region is detected from the input thermal image of the target person based on a first object detection algorithm and the at least one facial region is detected as the region of interest within the detected face region based on a second object detection algorithm.
